(19) United States
(12) Patent Application Publication
(10) Pub. No.: US 2024/0197252 A1
(43) Pub. Date: Jun. 20, 2024

(54) BIOLOGICAL INFORMATION MEASUREMENT DEVICE

(71) Applicant: OMRON HEALTHCARE CO., LTD., Muko-shi (JP)

(72) Inventors: Kentaro MORI, Kyoto (JP); Takeshi Kubo, Kyoto (JP); Takashi Ono, Kyoto (JP); Yoshihide Tokko, Kyoto (JP); Masaki Harada, Kyoto (JP); Kosuke Abe, Kyoto (JP); Shohei Iwata, Kyoto (JP); Yoshihiko Sano, Kyoto (JP)

(73) Assignee: OMRON HEALTHCARE CO., LTD., Muko-shi (JP)

(21) Appl. No.: 18/592,580

(22) Filed: Mar. 1, 2024

Related U.S. Application Data
(63) Continuation of application No. PCT/JP2023/004820, filed on Feb. 13, 2023.

(30) Foreign Application Priority Data
Jul. 22, 2022 (JP) 2022-117328

(51) Int. Cl. A61B 5/00; A61B 5/0235; A61B 5/33

(52) U.S. Cl. CPC A61B 5/6844; A61B 5/0235; A61B 5/33; A61B 5/681

(57) ABSTRACT

This biological information measurement device comprises: a fluid bag; a pump; a valve; a blood pressure measurement unit that measures the blood pressure; a first electrode that comes into contact with a first part of a subject; a second electrode that comes into contact with a second part, the second electrode being connected to the fluid bag, supported by a support member that extends farther in the circumferential direction from the circumferential end of the fluid bag, and positioned a predetermined distance away from the end in the circumferential direction; an electrocardiogram measurement unit that measures an electrocardiogram waveform of the subject; a belt part that is wrapped around the outer circumference of the fluid bag; and a contact state stabilizer for suppressing a change in the state of contact of the second electrode with respect to the second part due to a change in volume of the fluid bag.

### CROSS-REFERENCE TO RELATED APPLICATION

This application is the U.S. national stage application filed pursuant to 35 U.S.C. 365(c) and 120 as a continuation of International Patent Application No. PCT/JP2023/004820, filed on Feb. 13, 2023, which application claims priority to Japanese Patent Application No. 2022-117328, filed on Jul. 22, 2022, which applications are incorporated herein by reference in their entireties.

### TECHNICAL FIELD

The present invention relates to a biological information measurement device.

### BACKGROUND ART

In recent years, it has been common for an individual to measure his/her body/health information (hereinafter, also referred to as biological information) such as a blood pressure value and an electrocardiographic waveform by using a measurement instrument on a daily basis and utilize the measurement result for health management. For this reason, there is an increasing demand for instruments focusing on portability, many portable measurement devices have been proposed, and a portable instrument capable of measuring both the blood pressure value and the electrocardiographic waveform has also been proposed (see Patent Literatures 1 and 2).

In Patent Literature 1, an electrode is arranged on a back surface (a surface in contact with a user's body) of a main body of a wristwatch-type electrocardiograph.

In Patent Literature 2, an electrode is arranged on a front surface (a surface in contact with a user's body) of a belt-shaped cuff wound around an arm of the user.

### CITATION LIST

### Patent Literatures

Patent Literature 1: JP 2017-6230 A

Patent Literature 2: JP 2014-36843 A

### SUMMARY OF INVENTION

### Technical Problem

However, in a configuration in which the electrode is arranged on the back surface of the main body as in the technique described in Patent Literature 1, the cuff cannot be arranged on the back surface of the main body, and thus, a compressing force on the user for blood pressure measurement is insufficient. As in the technique described in Patent Literature 2, in a configuration in which the electrode is arranged on the surface of the cuff, it is difficult to wire the electrodes, and it is necessary to design the wire in consideration of the movement of the electrode accompanying the inflation of the cuff, resulting in a complicated configuration in design. In addition, in a case where a rigid electrode is used, the compression of an air bladder provided in the cuff is hindered.

In view of the above-described conventional techniques, an object of the present invention is to provide a biological information measurement device capable of stably measuring biological information with a simple configuration.

### Solution to Problem

In order to solve the above problems, the present invention provides a biological information measurement device which measures a blood pressure and an electrocardiographic waveform of a subject, the biological information measurement device including:
a fluid bag which is wound around a measurement target site of the subject in a circumferential direction;
a pump which supplies a fluid into the fluid bag;
a valve which communicates with the fluid bag and is provided in a flow path of the fluid;
a blood pressure measurement unit which compresses the measurement target site by supplying the fluid from the pump to inflate the fluid bag or releases compression on the measurement target site by controlling the valve to discharge the fluid in the fluid bag to contract the fluid bag, and measures a blood pressure of the measurement target site;
a first electrode which comes into contact with a first site of the subject;
a second electrode which comes into contact with a second site of the subject different from the first site, is connected to the fluid bag extending in the circumferential direction, is supported by a support member further extending in the circumferential direction from an end portion of the fluid bag in the circumferential direction, and is arranged at a position separated from the end portion by a predetermined distance in the circumferential direction;
an electrocardiogram measurement unit which measures an electrocardiographic waveform of the subject through the first electrode and the second electrode;
a belt portion which is wound around an outer circumferential side of the fluid bag and fixes the biological information measurement device to the measurement target site; and
contact state stabilizer which suppresses a change in a contact state of the second electrode with the second site accompanying a volume change of the fluid bag.

Accordingly, in a case where when the fluid bag wound around the measurement target site of the subject in the circumferential direction is inflated for blood pressure measurement of the subject, the first electrode to be used for measuring the electrocardiographic waveform of the subject and the second electrode in contact with the second site different from the first site of the subject with which the first electrode is in contact are connected to the fluid bag extending in the circumferential direction, are supported by the support member further extending in the circumferential direction from the end portion of the fluid bag in the circumferential direction, and are arranged at a position separated from the end portion by a predetermined distance in the circumferential direction, the support member moves toward the outer diameter side along with the inflation of the fluid bag, so that the posture (contact angle) of the second electrode with respect to the second site of the subject may change, the contact position may deviate, or the contact area may change. Such a change in the contact state between the second electrode and the second site affects stable measurement of the electrocardiographic waveform. In this regard, the contact state stabilizer which suppresses the change in the contact state of the second electrode with the second site accompanying the volume change of the fluid bag is provided so that stable measurement of the electrocardiographic waveform can be realized with a simple configuration.

In addition, in the present invention,
the contact state stabilizer may be an insulating covering portion which covers the second electrode and has an opening portion through which a part of the second electrode is exposed, and
a part of the second electrode exposed from the opening portion may be in contact with the second site before and after the volume change of the fluid bag.

Accordingly, stable measurement of the electrocardiographic waveform can be realized with a simple configuration in which the second electrode is covered with the insulating covering portion so as to be partially exposed.

In addition, in the present invention, the contact state stabilizer may be the support member which supports the second electrode at the position which allows a change in the contact state between the second electrode and the second site due to movement of the support member accompanying the volume change of the fluid bag to be limited to a predetermined range.

Accordingly, stable measurement of the electrocardiographic waveform can be realized with a simple configuration in which the second electrode is arranged at a position, which is separated by a predetermined distance from the end portion of the fluid bag in the circumferential direction, by the support member connected to the fluid bag, so as to allow the change in the contact state between the second electrode and the second site due to the movement of the support member accompanying the volume change of the fluid bag to be limited to a predetermined range.

In addition, in the present invention, the support member may be the support member including a second electrode support portion which supports the second electrode and a hinge portion which rotatably supports the second electrode support portion in a direction orthogonal to the circumferential direction.

In this way, with respect to the movement of the support member accompanying the inflation of the fluid bag, the degree of freedom of the second electrode support portion in the direction orthogonal to the circumferential direction can be obtained by the hinge portion, and thus the change in the contact state of the second electrode with the second site can be suppressed with a simple configuration of the hinge portion and the second electrode support portion.

In addition, in the present invention, the support member may constitute a part of the belt portion.

As described above, when the support member which supports the second electrode is constituted by a part of the belt portion, it is not necessary to provide a special member for supporting the second electrode, and the number of parts can be reduced.

In addition, in the present invention, a cross-sectional shape of the second electrode in a direction orthogonal to the circumferential direction may be a semicircle, an ellipse, an oval, or a curve protruding toward the second site.

As described above, when the cross-sectional shape of the second electrode in the direction orthogonal to the circumferential direction is formed into a semicircle, an ellipse, an oval, or a curve protruding toward the second site, it is possible to suppress a drastic change in the contact state between the second electrode and the second site accompanying the inflation of the fluid bag.

In addition, in the present invention, the fluid bag and the belt portion may be integrally provided.

In this way, the configuration of the biological information measurement device can be simplified, and handling is also facilitated.

In addition, in the present invention, the biological information measurement device may further include a curler which includes the support member and is curved along the circumferential direction of the measurement target site,
in which the curler and the belt portion may be integrally provided.

In this way, when the belt portion is provided integrally with the curler, the belt portion can be held in a curved shape following the circumferential direction of a measurement target part, the configuration of the biological information measurement device can be simplified, and handling is also facilitated.

In addition, in the present invention, the biological information measurement device may further include a third electrode which comes into contact with a third site of the subject and sets a reference potential,
in which the third electrode may be supported via an insulating member by the support member together with the second electrode, and
the contact state stabilizer may suppress a change in the contact state between the third electrode and the third site.

Providing the third electrode in this manner enables more stable measurement of the electrocardiographic waveform.

In addition, in the present invention, the biological information measurement device may further include:
a contact resistance measurement unit which measures a contact resistance against a body of the subject by the second electrode and the first electrode or a third electrode which comes into contact with a third site of the subject and sets a reference potential; and
a winding method determination unit which determines appropriateness of a winding method of the belt portion by the subject on the basis of the contact resistance.

Accordingly, even before the blood pressure measurement is started, the appropriateness of the winding method of the belt portion can be determined by using the second electrode and the first electrode or the third electrode for electrocardiographic waveform measurement, which is highly convenient.

### Advantageous Effects of Invention

According to the present invention, it is possible to provide a biological information measurement device capable of stably measuring biological information with a simple configuration.

### BRIEF DESCRIPTION OF DRAWINGS

FIG. 1 is a view illustrating an appearance of a biological information measurement device according to a first embodiment.

FIG. 2 is a view illustrating an appearance when the biological information measurement device according to the first embodiment is worn.

FIG. 3 is a functional block diagram of the biological information measurement device according to the first embodiment.

FIG. 4 is a cross-sectional view of a cuff assembly of the biological information measurement device according to the first embodiment.

FIGS. 5A and 5B are views illustrating a use state of the biological information measurement device according to the first embodiment.

FIGS. 6A and 6B are views illustrating a connection structure of electrodes according to the first embodiment.

FIGS. 7A and 7B are views illustrating the connection structure of the electrodes according to the first embodiment.

FIGS. 8A to 8C are views illustrating a configuration of the electrodes according to a first modification of the first embodiment.

FIG. 9 is a view illustrating a use state of a biological information measurement device according to a second modification of the first embodiment.

FIG. 10 is a view illustrating a use state of a biological information measurement device according to a second embodiment.

FIG. 11A is a view illustrating a configuration of electrodes of a biological information measurement device according to a third embodiment, and FIGS. 11B and 11C are views illustrating a use state of the biological information measurement device according to the third embodiment.

FIG. 12 is a diagram illustrating a configuration of a biological information measurement device according to a fourth embodiment.

FIG. 13 is a functional block diagram of a biological information measurement device according to a fifth embodiment.

FIG. 14 is a flowchart of winding method determination processing of the biological information measurement device according to the fifth embodiment.

### DESCRIPTION OF EMBODIMENTS

Hereinafter, specific embodiments of the present invention will be described on the basis of the drawings.

### First Embodiment

Hereinafter, an embodiment of an embodiment of the present invention will be described. However, the dimensions, materials, shapes, relative arrangements, and the like of the components described in this embodiment are not intended to limit the scope of the present invention only to them unless otherwise specified.

### Overall Configuration of Device

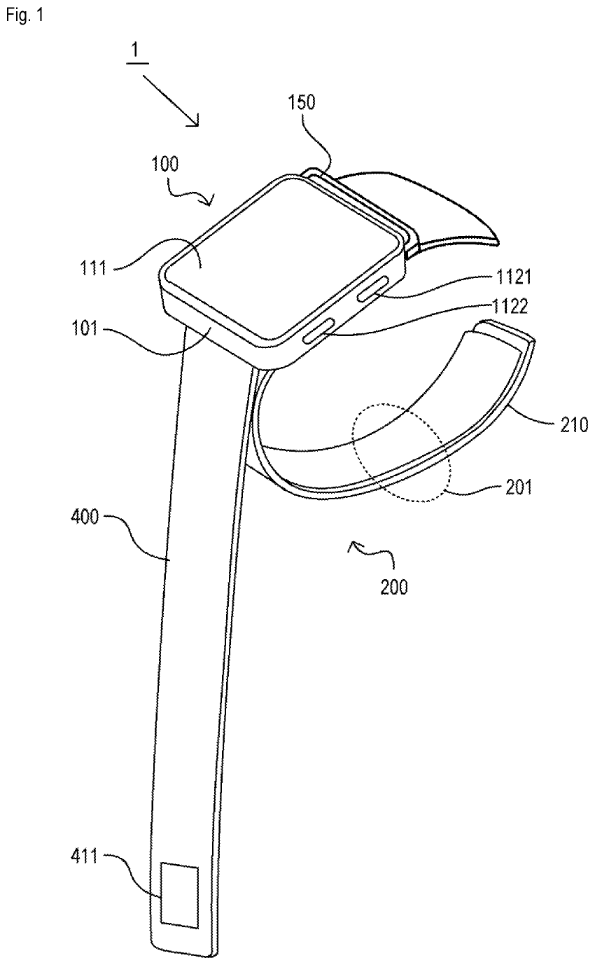

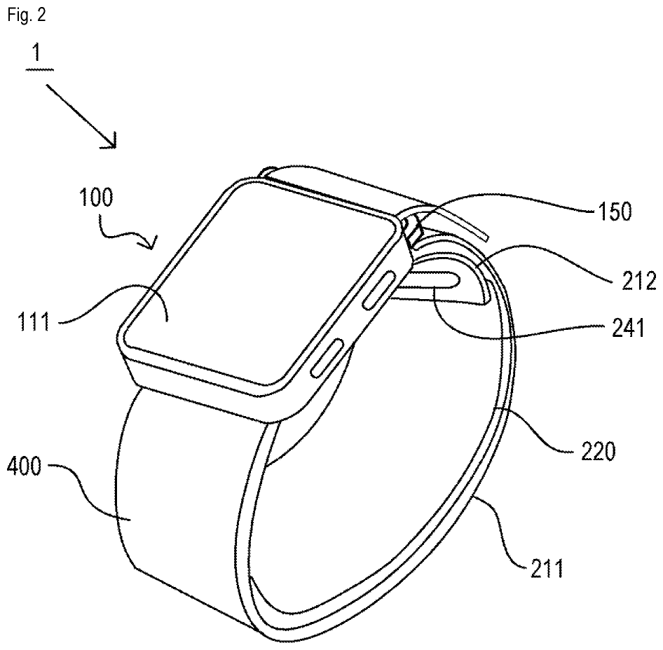

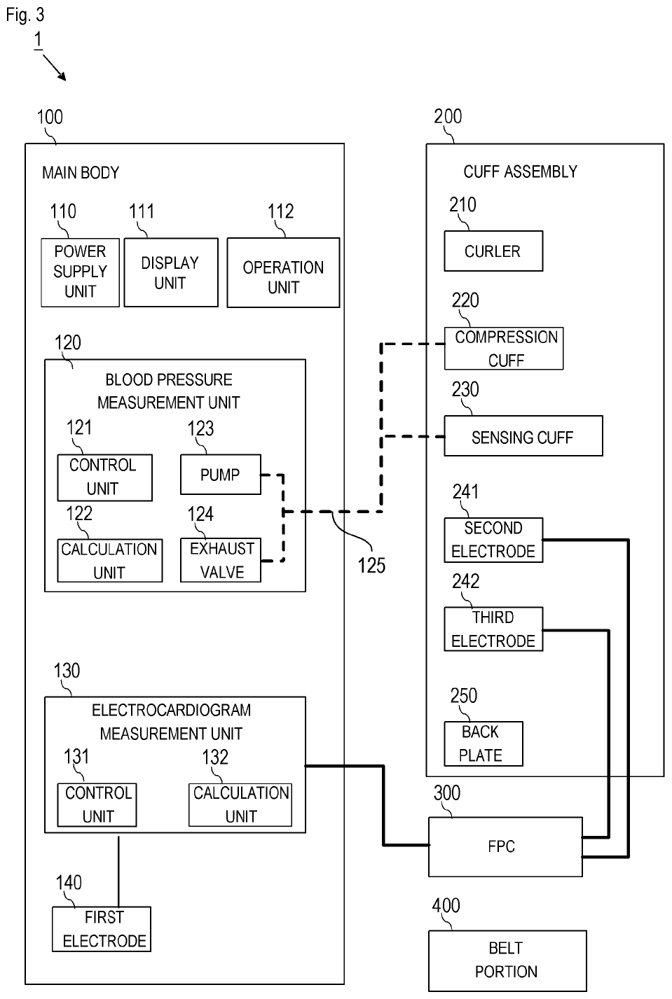

FIGS. 1 and 2 are schematic views illustrating an external configuration of a biological information measurement device 1 according to the present embodiment. FIG. 3 is a functional block diagram illustrating a functional configuration of the biological information measurement device 1 according to the present embodiment.

As illustrated in FIGS. 1 to 3, the biological information measurement device 1 schematically has a configuration which includes a main body 100, a cuff assembly 200, and a belt portion 400, and can measure a blood pressure value and an electrocardiographic waveform in the state of being worn on a wrist T of a subject. The belt portion 400 includes a hook-and-loop fastener 411 including a hook. The main body 100 is provided with a belt passing portion 150 including an annular belt passing ring for inserting the belt portion 400. When the biological information measurement device 1 is worn, the belt portion 400 is wound around the wrist T and then inserted into the belt passing portion 150, and the hook-and-loop fastener 411 is attached to an arbitrary position of the belt portion 400 (a loop with which a hook is engaged is formed) to perform fixing. In addition, the biological information measurement device 1 includes flexible printed circuits (FPCs) 300 (not illustrated in FIGS. 1 and 2) in which wiring for electrically connecting an electrocardiogram measurement unit 130 of the main body 100 to a second electrode 241 and a third electrode 242 of the cuff assembly 200 is arranged. Here, the wrist corresponds to a measurement target site of the present invention.

As illustrated in FIG. 3, the main body 100 includes a housing 101, a power supply unit 110, a display unit 111, an operation unit 112, a blood pressure measurement unit 120, the electrocardiogram measurement unit 130, and a first electrode 140. Here, the first electrode 140 includes the entire housing 101 of the main body 100 and operation buttons 1121 and 1122. The configuration of the first electrode 140 is not limited thereto, and may be a part of the housing 101 or may be a structure independent of the housing 101.

The power supply unit 110 includes a battery which supplies power necessary for operating the device. For example, the battery may be a secondary battery such as a lithium ion battery, or may be a primary battery.

The display unit 111 includes a display device such as a liquid crystal display, and may include an LED indicator and the like. Specifically, the operation unit 112 includes the operation buttons 1121 and 1122 arranged on the side surface of the housing 101 of the main body 100. A configuration such as a touch panel display may be made in which the display unit 111 and the operation unit 112 are integrated.

The blood pressure measurement unit 120 is a functional unit that controls the cuff assembly 200 to be described later and measures the blood pressure of the user on the basis of information obtained by the cuff assembly, and includes a control unit 121, a calculation unit 122, a pump 123, and an exhaust valve 124. The control unit 121 and the calculation unit 122 are configured by, for example, a central processing unit (CPU) or the like, and may include a storage unit (not illustrated) configured by a random access memory (RAM) or the like. Here, the pump 123 and the exhaust valve 124 correspond to a pump and a valve of the present invention, respectively.

The control unit 121 is a functional unit which controls the blood pressure measurement unit 120, controls the cuff pressure of the cuff assembly 200 via the calculation unit 122, the pump 123, and the like, and acquires information for measuring the blood pressure of the user from the artery on the wrist T on which the biological information measurement device 1 is worn. The calculation unit 122 measures the blood pressure value on the basis of the information acquired in this manner. The pump 123 and the exhaust valve 124 are mechanisms which communicate with a compression cuff 220 and a sensing cuff 230 to be described later via a flow path 125 through which air flows, and are responsible for supplying and discharging air to and from the compression cuff 220 and the sensing cuff 230.

The electrocardiogram measurement unit 130 is a functional unit which measures an electrocardiographic waveform of the user on the basis of a potential difference between the first electrode 140 and second electrode 241 in contact with a human body surface, and includes a control unit 131 and a calculation unit 132. The control unit 131 and the calculation unit 132 are configured by the above-described CPU and the like. From the viewpoint of hardware, the control unit 131 and the calculation unit 132 may have a configuration common to the control unit 121 and the calculation unit 122 of the blood pressure measurement unit 120.

Note that both the blood pressure measurement unit 120 and the electrocardiogram measurement unit 130 include an AD conversion circuit, an amplifier, a filter, and the like (not illustrated) in addition to the CPU, RAM, and the like described above, but since these are configured by known techniques, the description thereof is omitted.

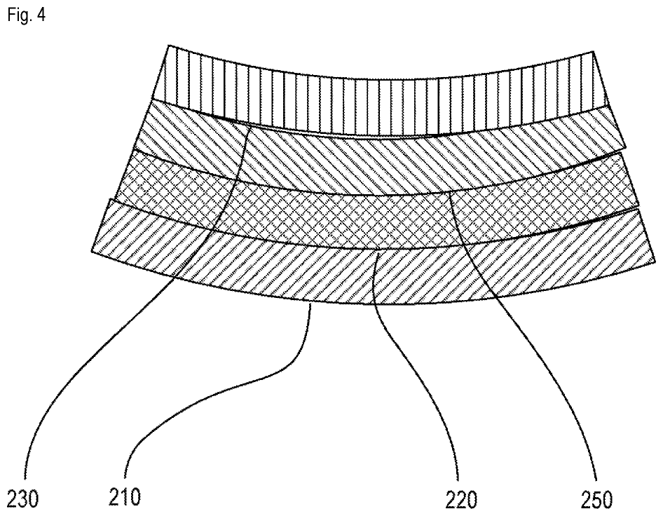

The cuff assembly 200 includes a curler 210, the compression cuff 220, the sensing cuff 230, the second electrode 241, the third electrode 242, and a back plate 250. The curler 210 is a member serving as a base for holding the compression cuff 220. FIG. 4 is a cross-sectional view schematically illustrating an internal structure of an area, which is surrounded by a dotted line in FIG. 1, in the cuff assembly 200. The cuff assembly 200 has a configuration in which the compression cuff 220, the back plate 250, and the sensing cuff 230 are laminated in this order with the curler 210 as the outermost side. Here, the compression cuff 220 (and the sensing cuff 230) corresponds to a fluid bag of the present invention.

The compression cuff 220 has a role of tightening the wrist T of the wearing portion by being inflated by the air sent from the pump 123 and applying an external pressure to an artery (not illustrated) present on the wrist T. In addition, the sensing cuff 230 (not illustrated) is a fluid bag for detecting the pressure applied to the site compressed by the compression cuff 220, and detects, with a small amount of air in the sensing cuff 230, the internal pressure thereof by using a pressure gauge (not illustrated), so as to measure the pressure applied to the compression site. In addition, the back plate 250 (not illustrated) is a flexible flat plate member arranged between the compression cuff 220 and the sensing cuff 230, suppresses excessive bending of the sensing cuff 220 at the time of compression by the compression cuff 230, and smooths the pressure distribution in the sensing cuff 230. Here, the air corresponds to a fluid of the present invention.

Both the second electrode 241 and the third electrode 242 are electrodes arranged at positions where the second electrode and the third electrode can be in contact with a human body surface, the second electrode 241 functions as an electrode for electrocardiographic waveform measurement, and the third electrode 242 functions as a ground (GND) electrode for setting a reference potential.

### Structure of Cuff Assembly Portion

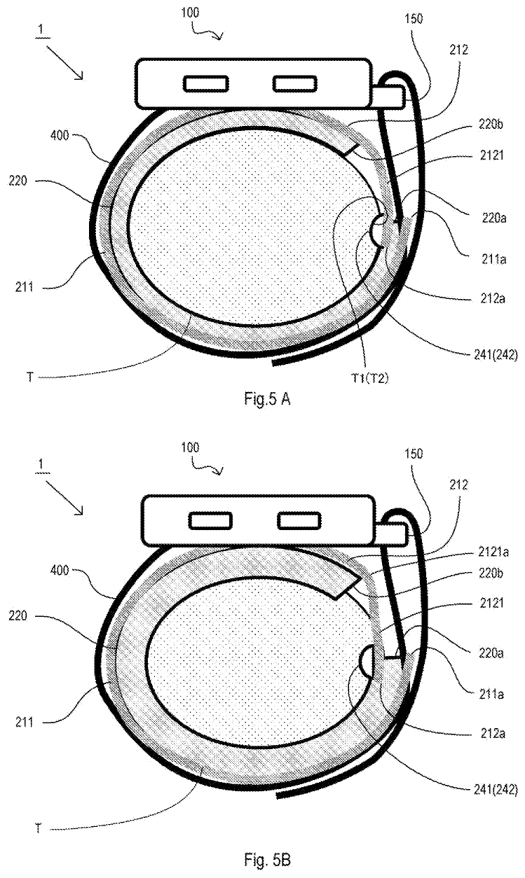

The structure of the cuff assembly 200 will be described on the basis of FIG. 5 illustrating a state where the biological information measurement device 1 is worn on the wrist T of the user. In the present embodiment, the compression cuff 220 is provided along the extending direction (a direction around the wrist T) of the curler 210 formed in a C shape curved following the circumferential direction of the wrist T. The curler 210 includes a first curler portion 211 longer in the extending direction and a second curler portion 212 shorter in the extending direction with reference to the position where the main body 100 is provided. The first curler portion 211 extends from the main body 100 positioned on the back side of the wrist T so as to cover the artery side of the wrist T. On the other hand, the second curler portion 212 extends to the side opposite to the first curler portion 211 with respect to the circumferential direction of the wrist T. The compression cuff 220 is provided along the first curler portion 211 of the curler 210, and a distal end portion 220a of the compression cuff 220 in the circumferential direction (the extending direction of the first curler portion 211) is positioned in the vicinity of a distal end portion 211a of the first curler portion 211 in the extending direction. In addition, the compression cuff 220 is also provided continuously from the first curler portion 211 side along the second curler portion 212, but a distal end portion 220b of the compression cuff 220 in the circumferential direction (the extending direction of the second curler portion 212) is positioned away from a distal end portion 212a of the second curler portion 212 of the curler 210 in the extending direction. The curler 210 corresponds to a curler of the present invention.

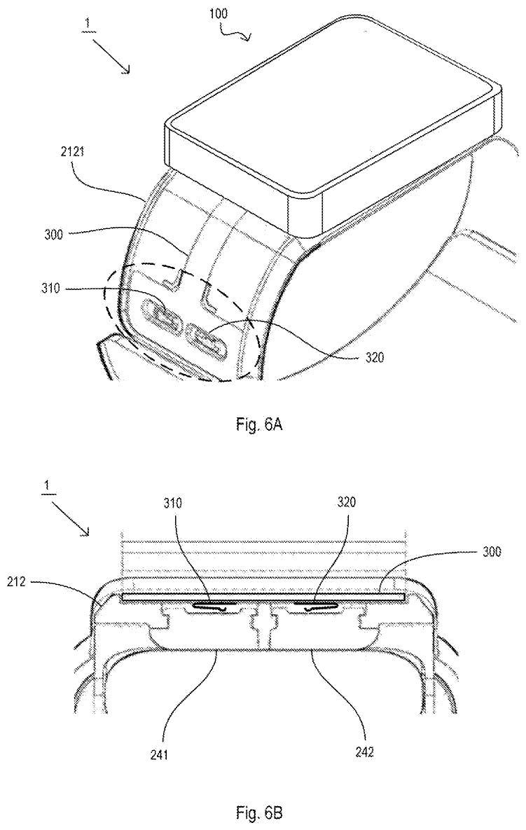

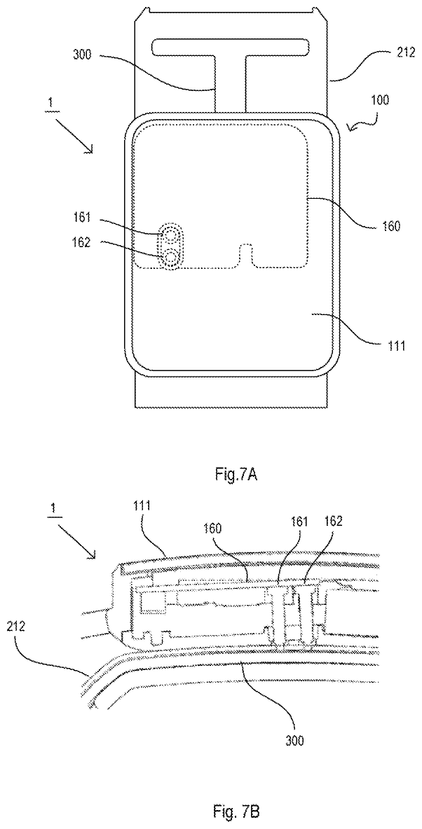

As described above, an electrode support portion 2121 in which the compression cuff 220 is not provided inside is provided on the distal end side of the second curler portion 212 of the curler 210. FIG. 6A is a perspective view of the biological information measurement device 1 as viewed from the outside of the electrode support portion 2121, and FIG. 6B is a cross-sectional view of a portion surrounded by a broken line in FIG. 6A as viewed from a direction orthogonal to the circumferential direction. The second electrode 241 and the third electrode 242 are arranged inside the electrode support portion 2121. The second electrode 241 and the third electrode 242 are made of a conductive member, such as stainless steel, having a substantially semicircular cross section orthogonal to the circumferential direction and a shape, which is obtained by dividing an oval shape into two, in the circumferential direction, and are arranged at predetermined intervals in a direction orthogonal to the circumferential direction. As illustrated in FIG. 6A, the FPC 300 is arranged from the main body 100 toward the second electrode 241 and the third electrode 242 along the second curler portion 212 of the curler 210. The end portion of the FPC 300 in the circumferential direction (the extending direction of the second curler portion 212) is connected to the second electrode 241 and the third electrode 242 by contact pins 310 and 320 in the form of a leaf spring, respectively. FIG. 7A is a view illustrating a part of the internal structure of the main body 100 when viewed from the front of the display unit 111 of the biological information measurement device 1, and FIG. 7B is a view illustrating a connection structure between a substrate 160 and the FPC 300 in a cross section passing through the centers of pogo pins 161 and 162. As illustrated in FIGS. 7A and 7B, the substrate 160 accommodated in the main body 100 and the FPC 300 are electrically connected by the pogo pins 161 and 162. In FIGS. 6A, 6B, 7A, and 7B, the illustration of configurations unnecessary for description are appropriately omitted.

As described with reference to FIG. 5A, in the present embodiment, the second electrode 241 and the third electrode 242 are arranged at positions beyond the distal end portion 220b of the compression cuff 220 in the circumferential direction (the extending direction of the second curler portion 212) and separated from the distal end portion 220b by a predetermined distance. FIG. 5A illustrates a state where the belt portion 400 is wound around the wrist T on the outer circumferential side of the curler 210, inserted into the belt passing portion 150, and fixed by the hook-and-loop fastener 411, and the compression cuff 220 is not inflated, and FIG. 5B illustrates a state after the compression cuff 220 is inflated. As described above, when the compression cuff 220 arranged inside is inflated, the curler 210 having a C shape is expanded and moved to an outer diameter side. However, in the biological information measurement device 1 according to the present embodiment, the second electrode 241 and the third electrode 242 are provided in the electrode support portion 2121 extending beyond the distal end portion 220b of the inflating compression cuff 220 in the circumferential direction (the extending direction of the second curler portion 212). Therefore, even when a proximal end portion 2121a of the electrode support portion 2121 moves to the outer diameter side along with the inflation of the compression cuff 220, the movement and posture change of the second electrode 241 and the third electrode 242 provided at the position separated from the proximal end portion 2121a by a predetermined distance are limited to a predetermined range, so that the change in the contact state of the second electrode 241 and the third electrode 242 with the wrist T is suppressed. Here, the change in the contact state is a change in the contact state such as a contact position, a contact angle, and a contact area of the second electrode 241 and the third electrode 242 with the wrist T. In FIGS. 4A and 4B, since the compression cuff 220 arranged inside the distal end portion 211a of the first curler portion 211 of the curler 210 is positioned outside the electrode support portion 2121 of the curler 210, the electrode support portion 2121 is pressed toward the inner diameter side by the inflation of the compression cuff 220. Since a contact pressure of the second electrode 241 and the third electrode 242 against the wrist T also increases by the pressing of the compression cuff 220 toward the inner diameter side, the change in the contact state of the second electrode 241 and the third electrode 242 with the wrist T is further suppressed. Depending on the thickness of the wrist T, the distal end portion 220a of the compression cuff 220 in the circumferential direction (the extending direction of the first curler portion 211) may not reach the outer diameter side of the electrode support portion 2121. However, even in such a case, since the movement of the electrode support portion 2121 to the outer diameter side is suppressed by the belt portion 400 wound around the wrist T, the movement and posture change of the second electrode 241 and the third electrode 242 due to the inflation of the compression cuff 220 are suppressed, and the change in the contact state of the second electrode 241 and the third electrode 242 with the wrist T is suppressed. Here, the electrode support portion 2121 corresponds to a support member and contact state stabilizer of the present invention. The inflation of the compression cuff 220 corresponds to a volume change of the fluid bag of the present invention.

In the shapes of the second electrode 241 and the third electrode 242, a cross-sectional shape in the direction orthogonal to the circumferential direction is not limited to a semicircular shape as illustrated in FIG. 5, and may be a semi-elliptical shape, a semi-oval shape, or another curved shape such as a curve protruding toward the wrist T.

### Measurement of Biological Information

In order to measure the biological information by the biological information measurement device 1 having the above configuration, first, the cuff assembly 200 and the belt portion 400 are wound around the wrist T such that the main body 100 faces the back side of the hand. Then, the belt portion 400 is passed through the belt passing portion 150 and folded back, the hook-and-loop fastener 411 of the belt portion 400 is stuck to an arbitrary position of the belt portion 400, and the biological information measurement device 1 is worn on the wrist T. At this time, the sensing cuff 230 is worn to be positioned on the palm side of the wrist T.

Then, the measurement is started by operating the operation button 1121 (or 1122). Specifically, the compression cuff 220 is inflated by injecting air into the compression cuff 220, so as to compress (the artery of) the wrist T, obstruct the artery, and temporarily stop blood flow, then the compression cuff 220 is contracted by gradually discharging the air from the compression cuff 220, so as to release the compression and restore the blood flow in the artery, and the pressure at that time is measured by the sensing cuff 230. That is, the blood pressure measurement by a so-called oscillometric method is performed.

Then, during the blood pressure measurement, when the wrist T is compressed by the compression cuff 220, the second electrode 241 and the third electrode 242 come into contact with (are pressed against) surfaces T1 and T2 (see FIG. 5A) of the wrist T. Therefore, when the first electrode 140 provided in the housing 101 of the main body 100 is touched with the finger on which the biological information measurement device 1 is not worn, the electrocardiographic waveform can be measured by a so-called I induction method on the basis of the potential difference between the first electrode 140 and the second electrode 241. Here, the finger on which the biological information measurement device 1 is not worn corresponds to a first site of the present invention, and the surfaces T1 and T2 of the wrist T correspond to a second site and a third site of the present invention, respectively.

As described above, according to the biological information measurement device 1 of the present embodiment, the blood pressure value and the electrocardiographic waveform can be simultaneously and accurately measured by a portable device of the type worn on the wrist T.

### First Modification

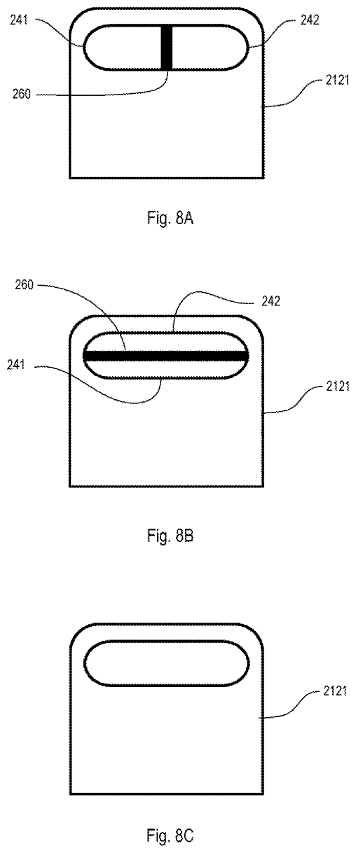

FIGS. 8A and 8B are views of the electrode support portion 2121 of the curler 210 provided with the second electrode 241 and the third electrode 242 as viewed from the inside.

In the first embodiment, the second electrode 241 and the third electrode 242 are arranged apart from each other, but as illustrated in FIG. 8A, a separator 260 made of an insulating member may be arranged between the second electrode 241 and the third electrode 242. As described above, by arranging the second electrode 241 and the third electrode 242 with the separator 260 interposed therebetween, the second electrode 241 and the third electrode 242 can be insulated from each other, and an interval between the second electrode 241 and the third electrode 242 can be reduced. At this time, in FIG. 5A, the second electrode 241 is arranged on the left side and the third electrode 242 is arranged on the right side, but the positions of the second electrode 241 and the third electrode 242 may be exchanged, and the second electrode 241 and the third electrode 242 may be arranged on the right side and the left side, respectively. Here, the separator 260 corresponds to an insulating member of the present invention.

The arrangement direction of the second electrode 241 and the third electrode 242 is not limited to the direction orthogonal to the circumferential direction. As illustrated in FIG. 8B, the second electrode 241 may be arranged on the proximal end side (the lower side in FIG. 8B) and the third electrode 242 may be arranged on the distal end side along the circumferential direction, or a separator made of an insulating member may be arranged between the second electrode 241 and the third electrode 242.

In addition, FIG. 8C illustrates a modification in which the third electrode 242 is omitted and only the second electrode 241 is provided in the electrode support portion 2121. As described above, by omitting the third electrode 242 functioning as the GND electrode, it is possible to reduce the number of members and simplify the structure.

### Second Modification

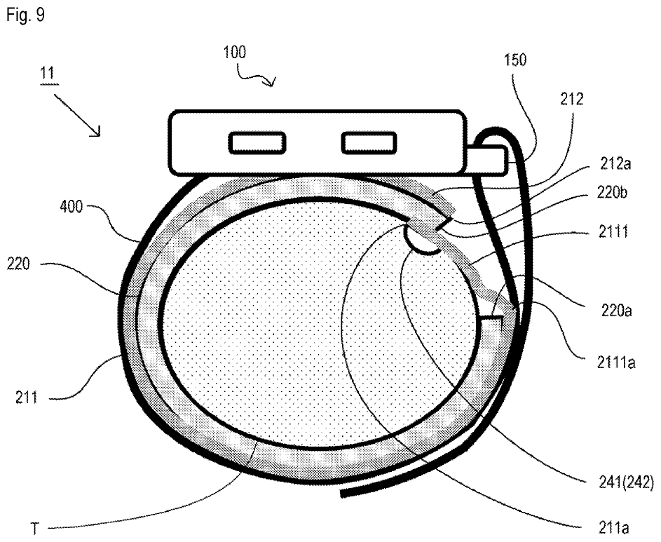

FIG. 9 is a side view illustrating a configuration of a biological information measurement device 11 according to a second modification of the first embodiment. The same configurations as those of the biological information measurement device 1 according to the first embodiment are denoted by the same reference numerals, and detailed description thereof will be omitted.

The biological information measurement device 11 has the same configuration as the biological information measurement device 1 except for the arrangement of the second electrode 241 and the third electrode 242.

In the biological information measurement device 1, the second electrode 241 and the third electrode 242 are arranged in the electrode support portion 2121 provided on the distal end side of the second curler portion 212 of the curler 210, but in the biological information measurement device 11 according to the second modification, the second electrode 241 and the third electrode 242 are arranged in an electrode support portion 2111 provided on the distal end side of the first curler portion 211 of the curler 210.

In the biological information measurement device 11, the distal end portion 220a of the compression cuff 220 in the circumferential direction (the extending direction of the first curler portion 211) is positioned at a predetermined distance from the distal end portion 211a of the curler 210 in the extending direction. Then, the first curler portion 211 of the curler 210 extends beyond the distal end portion 220a of the compression cuff 220, which is arranged inside the curler 210 along the first curler portion 211, in the circumferential direction (the extending direction of the first curler portion 211), and the electrode support portion 2111 is provided at a position of the curler 210 beyond the distal end portion 220a of the compression cuff 220. The second electrode 241 and the third electrode 242 are arranged inside the electrode support portion 2111. Also in the biological information measurement device 11, the second electrode 241 and the third electrode 242 are made of a conductive member, such as stainless steel, having a substantially semicircular cross section in the circumferential direction and an oval shape in a direction orthogonal to the circumferential direction, and are arranged side by side in the direction orthogonal to the circumferential direction. Although not illustrated, the second electrode 241 and the third electrode 242 are electrically connected to the substrate 160 accommodated inside the main body 100 via the FPC 300.

As described above, in the second modification, the second electrode 241 and the third electrode 242 are arranged at positions beyond the distal end portion 220a of the compression cuff 220 in the circumferential direction (the extending direction of the first curler portion 211) and separated from the distal end portion 220a by a predetermined distance. FIG. 9 illustrates a state where the belt portion 400 is wound around the wrist T on the outer circumferential side of the curler 210, inserted into the belt passing portion 150, and fixed by the hook-and-loop fastener 411, and the compression cuff 220 is not inflated. When the compression cuff 220 arranged inside is inflated, as described with reference to FIGS. 5A and 5B, the curler 210 having a C shape is expanded and moved to the outer diameter side. However, in the biological information measurement device 11 according to the second modification, the second electrode 241 and the third electrode 242 are provided in the electrode support portion 2111 extending beyond the distal end portion 220a of the inflating compression cuff 220 in the circumferential direction (the extending direction of the first curler portion 211). Therefore, even when a proximal end portion 2111a of the electrode support portion 2111 moves to the outer diameter side along with the inflation of the compression cuff 220, the movement and posture change of the second electrode 241 and the third electrode 242 provided at the position separated from the proximal end portion 2111a by a predetermined distance are suppressed, so that the change in the contact state of the second electrode 241 and the third electrode 242 with the wrist T is suppressed. In FIG. 9, since the compression cuff 220 arranged at the distal end portion 212a of the second curler portion 212 of the curler 210 is positioned on the outer diameter side of the electrode support portion 2111 of the curler 210, the electrode support portion 2111 is pressed toward the inner diameter side by the inflation of the compression cuff 220. Since a contact pressure of the second electrode 241 and the third electrode 242 against the wrist T also increases by the pressing of the compression cuff 220 toward the inner diameter side, the contact state of the second electrode 241 and the third electrode 242 with the wrist T is further suppressed. Depending on the thickness of the wrist T, the distal end portion 220b of the compression cuff 220 in the circumferential direction (the extending direction of the second curler portion 212) may not reach the outer diameter side of the electrode support portion 2111. However, even in such a case, since the movement of the electrode support portion 2111 to the outer diameter side is suppressed by the belt portion 400 wound around the wrist T, the movement and posture change of the second electrode 241 and the third electrode 242 due to the inflation of the compression cuff 220 are limited to a predetermined range, and the change in the contact state of the second electrode 241 and the third electrode 242 with the wrist T is suppressed. Here, the electrode support portion 2111 corresponds to a support member and contact state stabilizer of the present invention.

Note that also in the second modification, the configuration of the first modification illustrated in FIGS. 8A to 8C may be applied to the configuration of the second electrode 241 and the third electrode 242.

### Second Embodiment

Hereinafter, a biological information measurement device 12 according to a second embodiment of the present invention will be described with reference to the drawings. The same configurations as those of the biological information measurement device 1 according to the first embodiment are denoted by the same reference numerals, and detailed description thereof will be omitted.

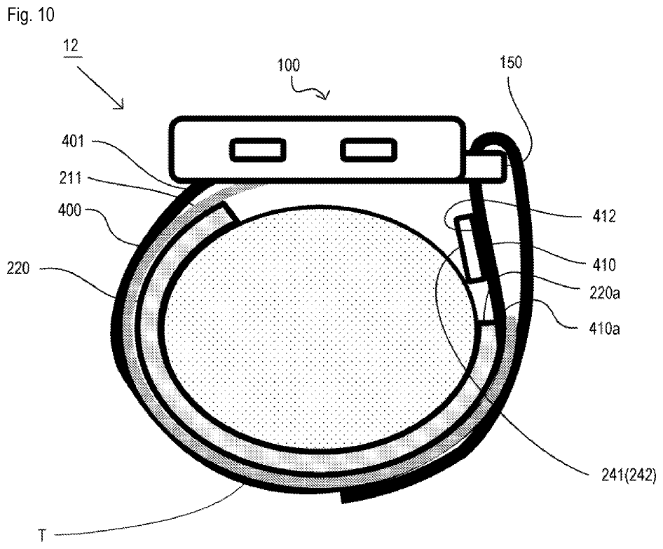

FIG. 10 is a side view illustrating a configuration of the biological information measurement device 12 according to the second embodiment.

In FIG. 10, in the biological information measurement device 12, only the first curler portion 211 extending from the main body 100 so as to cover the artery side of the wrist T is illustrated in the curler 210, but the second curler portion 212 may be provided which extends to the side opposite to the first curler portion 211 with respect to the circumferential direction of the wrist T and has an appropriate extension. In addition, in FIG. 10, the compression cuff 220 is provided only on the inner circumferential side of the first curler portion 211 with respect to the main body 100, but may be provided up to an appropriate position in the circumferential direction of the wrist T according to the configuration of the curler 210.

In the biological information measurement device 12, the second electrode 241 and the third electrode 242 are arranged on the inner circumferential surface 412 of the electrode support portion 410 on the side opposite to the end portion 401 of the belt portion 400 on the main body 100 side. The electrode support portion 410 of the belt portion 400 is provided at a position separated from the distal end portion 220a of the compression cuff 220 in the circumferential direction (the extending direction of the first curler portion 211) by a predetermined distance. The second electrode 241 and the third electrode 242 are electrically connected to the substrate 160 accommodated inside the main body 100 by the FPC provided on the belt portion 400.

FIG. 10 illustrates a state before the compression cuff 220 is inflated. When the compression cuff 220 is inflated, the curler 210 having a C shape is expanded and moved to the outer diameter side as described with reference to FIGS. 5A and 5B, so that the belt portion 400 and the electrode support portion 410 wound around the outer circumferential side of the curler 210 also move to the outer diameter side. However, in the biological information measurement device 12, the second electrode 241 and the third electrode 242 are provided in the electrode support portion 410 extending beyond the distal end portion 220a of the inflating compression cuff 220 in the circumferential direction (the extending direction of the first curler portion 211). Therefore, even when a proximal end portion 410a of the electrode support portion 410 moves to the outer diameter side along with the inflation of the compression cuff 220, the movement and posture change of the second electrode 241 and the third electrode 242 provided at the position separated from the proximal end portion 410a by a predetermined distance are limited to a predetermined range, so that the change in the contact state of the second electrode 241 and the third electrode 242 with the wrist T is suppressed.

Here, the electrode support portion 410 constituting a part of the belt portion 400 corresponds to a support member and contact state stabilizer of the present invention.

The present embodiment can also be combined with each embodiment described later. In addition, in the biological information measurement device 12 illustrated in FIG. 10, the curler 210 is provided, but it is allowed to have a configuration in which the curler 210 is omitted.

### Third Embodiment

Hereinafter, a biological information measurement device 13 according to a third embodiment of the present invention will be described with reference to the drawings. The same configurations as those of the biological information measurement device 1 according to the first embodiment are denoted by the same reference numerals, and detailed description thereof will be omitted.

In the biological information measurement device 13, the configurations of the electrode support portion 2122, the second electrode 241, and the third electrode 242 provided on the circumferential distal end side of the second curler portion 212 of the curler 210 are different from those of the biological information measurement device 1, and the other configurations are common.

Also in the biological information measurement device 13, the electrode support portion 2122 is provided on the distal end side of the second curler portion 212 of the curler 210 in the extending direction. In addition, the electrode support portion 2122 extends beyond the distal end portion 220b, in the circumferential direction, of the compression cuff 220 provided inside the curler 210. The second electrode 241 and the third electrode 242 are arranged inside a distal end portion 2122a of the electrode support portion 2122, but can be arranged at an appropriate position from the distal end portion 220a of the compression cuff 220 in the circumferential direction.

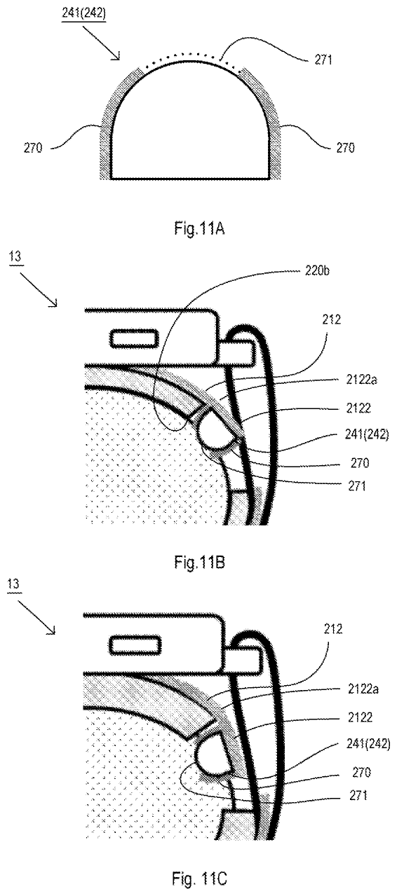

FIG. 11A is a view schematically illustrating a relationship between the second electrode 241 and the third electrode 242 according to the third embodiment, and an insulating film 270 in a cross section in a direction orthogonal to the circumferential direction. As illustrated in FIG. 11A, the proximal end side (the electrode support portion 2112 side or the outer side) of the second electrode 241 and the third electrode 242 is covered with the insulating film 270. Therefore, among the second electrode 241 and the third electrode 242, only a portion exposed from an opening portion 271 (which is indicated by a dotted line in FIG. 11A) formed by the insulating film 270 is electrically in contact with the wrist T. That is, the insulating film 270 has a function of limiting the contact portions of the second electrode 241 and the third electrode 242 with the wrist T. The range of the portion covered by the insulating film 270 and the opening portion 271 may vary depending on the shapes of the second electrode 241 and the third electrode 242, the circumferential length of the electrode support portion 2122, the thickness of the compression cuff 220 before the inflation and after the inflation, and the like, but the portions of the second electrode 241 and the third electrode 242 exposed from the opening portion 271 are set so as to be commonly in contact with the wrist T before and after the inflation of the compression cuff 220 in a state where the biological information measurement device 13 is worn on the wrist T. When the heights of the second electrode 241 and the third electrode 242 protruding inward from the electrode support portion 2122 are defined as from the proximal end side (electrode support portion 2122 side) to the inner side, the height from the proximal end side of the insulating film 270 may be different between the distal end side in the circumferential direction and the proximal end side (main body 100 side) in the circumferential direction, and for example, can be set so as to be low on the distal end side in the circumferential direction and high on the proximal end side in the circumferential direction.

FIG. 11B illustrates a state where the compression cuff 220, the curler 210, and the belt portion 400 are wound around the wrist T, the hook-and-loop fastener 411 of the belt portion 400 inserted into the belt passing portion 150 is fixed, and the compression cuff 220 is not inflated, and FIG. 11C illustrates a state where the compression cuff 220 is inflated from the state of FIG. 11B. As compared with the state illustrated in FIG. 11B before the compression cuff 220 is inflated, in the state illustrated in FIG. 11C in which the compression cuff 220 is inflated, a proximal end portion 2112a of the electrode support portion 2112 is pulled in the radially outward direction, and the second electrode 241 and the third electrode 242 rotate clockwise in the drawing. As described above, there is a possibility that the contact range of the second electrode 241 and the third electrode 242 with the wrist T changes before and after the inflation of the compression cuff 220. However, since the portion of the second electrode 241 and the third electrode 242 exposed from the opening portion 271 is electrically in contact with the wrist T in any state before and after the inflation of the compression cuff 220, the contact portion of the second electrode 241 and the third electrode 242 with the wrist T does not change due to the inflation of the compression cuff 220. That is, the portion of the second electrode 241 and the third electrode 242 which is in contact with the wrist T only at any stage in the inflation of the compression cuff 220 is covered with the insulating film 270, so that it is possible to achieve an insulated state where the portion is not in electrical contact with the wrist T. Here, the insulating film 270 and the opening portion 271 correspond to a covering portion and an opening portion of the present invention, respectively. In addition, the insulating film 270 and the opening portion 271 correspond to contact state stabilizer of the present invention.

By providing such an insulating film 270, the contact state of the second electrode 241 and the third electrode 242 with the wrist T can be stabilized regardless of the inflation of the compression cuff 220. In addition, by providing such an insulating film 270, the contact state of the second electrode 241 and the third electrode 242 with the wrist T can be stabilized even in a case where the second electrode 241 and the third electrode 242 are arranged at a position beyond the distal end portion 220a of the compression cuff 220 in the circumferential direction (the extending direction of the second curler portion 212) and close to the distal end portion 220a. However, the insulating film 270 in the third embodiment can also be applied to the second electrode 241 and the third electrode 242 of the biological information measurement device 1 according to the first modification of the first embodiment. In addition, the insulating film 270 in the third embodiment can also be applied to a case where the second electrode 241 and the third electrode 242 are provided in the electrode support portion 2112 extending to the distal end of the first curler portion 211 of the curler 210 as in the biological information measurement device 13 according to the second modification of the first embodiment.

### Fourth Embodiment

Hereinafter, a biological information measurement device 14 according to a fourth embodiment of the present invention will be described with reference to the drawings. The same configurations as those of the biological information measurement device 1 according to the first embodiment are denoted by the same reference numerals, and detailed description thereof will be omitted.

In the biological information measurement device 14 according to the present embodiment, the configuration of the electrode support portion 2123 of the curler 210 in which the second electrode 241 and the third electrode 242 are arranged is different from that of the biological information measurement device 1 according to the first embodiment, and the other configurations are common.

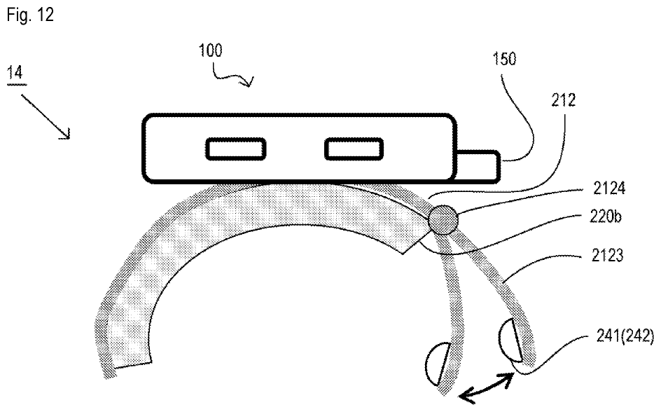

As illustrated in FIG. 12, the electrode support portion 2123 is connected to the distal end of the second curler portion 212 of the curler 210 by a hinge portion 2124 rotatable about a shaft in a direction orthogonal to the circumferential direction. As illustrated in FIGS. 5A and 5B, the electrode support portion 2121 of the curler 210 can be deformed by the inflation of the compression cuff 220. By connecting the electrode support portion 2123 to the second curler portion 212 of the curler 210 via the hinge portion 2124, deformation accompanying the inflation of the compression cuff 220 is allowed, so that it is possible to suppress the contact state of the second electrode 241 and the third electrode 242 with the wrist T from becoming unstable due to the rigidity of the electrode support portion 2123. Here, the electrode support portion 2123 corresponds to a support member and a second electrode support portion of the present invention, and the electrode support portion 2123 and the hinge portion 2124 correspond to contact state stabilizer of the present invention.

In the biological information measurement device 14 described above, the hinge portion 2124 is provided only at one position between the distal end of the second curler portion 212 of the curler 210 and the electrode support portion 2123. However, hinge portions which are similarly rotatable in the direction orthogonal to the circumferential direction may be provided at a plurality of positions of the curler 210 along the circumferential direction, or the hinge portions may be arranged over the entire curler 210 in the circumferential direction like a band of a wristwatch.

### Fifth Embodiment

Hereinafter, a biological information measurement device 15 according to a fifth embodiment of the present invention will be described with reference to the drawings. The same configurations as those of the biological information measurement device 1 according to the first embodiment are denoted by the same reference numerals, and detailed description thereof will be omitted.

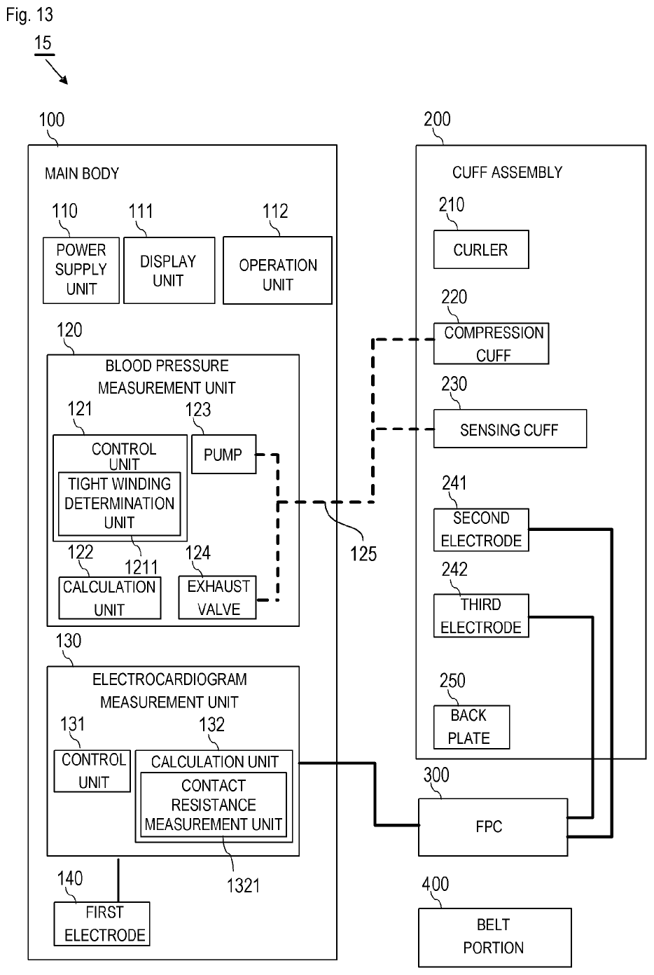

The biological information measurement device 15 according to the fifth embodiment has the same hardware configuration as those in the first to fourth embodiments. FIG. 13 illustrates a functional block diagram of the biological information measurement device 15 according to the present embodiment. The biological information measurement device 15 is different from the biological information measurement device 1 according to the first embodiment illustrated in FIG. 3 in the block configuration of the calculation unit 132 of the electrocardiogram measurement unit 130 and the control unit 121 of the blood pressure measurement unit 120. In the biological information measurement device 15, the calculation unit 132 includes a contact resistance measurement unit 1321, and the control unit 121 includes a belt tight winding determination unit 1221. The functions of the contact resistance measurement unit 1321 and the belt tight winding determination unit 1221 will be described later. Here, the contact resistance measurement unit 1321 and the belt tight winding determination unit 1221 correspond to contact resistance measurement unit and a winding method determination unit of the present invention, respectively.

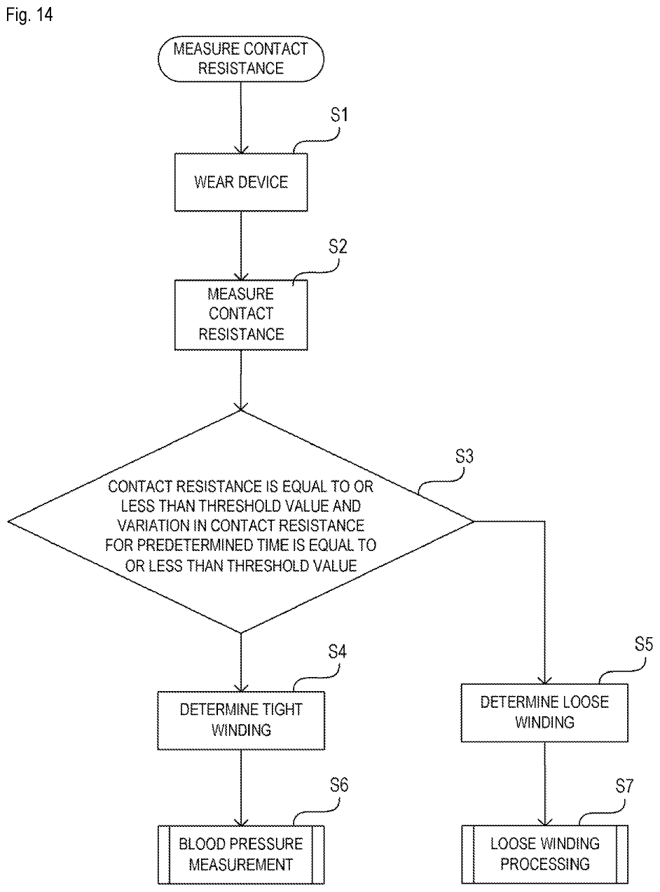

FIG. 14 is a flowchart illustrating a procedure of belt tight fitting determination processing in the biological information measurement device 15.

First, the user wears the biological information measurement device 15 and starts the blood pressure measurement (step S1). Specifically, the compression cuff 220, the curler 210, and the belt portion 400 are wound around the wrist T, the belt portion 400 is inserted into the belt passing portion 150, the belt portion 400 is fixed by the hook-and-loop fastener 411, a predetermined measurement posture is taken, and a measurement switch is pressed while the first electrode 140 is touched with the finger of the hand opposite to the hand on which the biological information measurement device 15 is worn.

Next, the contact resistance measurement unit 1321 measures a contact resistance between the second electrode 241 and the third electrode 242, and the wrist T by conduction between the second electrode 241 and the third electrode 242 (step S2). At this time, the contact resistance may be measured by conduction between the first electrode 140 and the second electrode 141. In addition, the site of the body of the subject where the contact resistance is measured is not limited to the wrist T.

Then, the belt tight winding determination unit 1221 determines whether or not the contact resistance is equal to or less than a threshold value and a variation in contact resistance for a predetermined time is equal to or less than a threshold value (step S3).

At this time, when the contact resistance is equal to or less than the threshold value and the variation in contact resistance for the predetermined time is equal to or less than the threshold value, the belt tight winding determination unit 1221 determines tight winding, that is, that the belt portion 400 is tightly wound around the wrist T and the biological information measurement device 15 is appropriately worn (step S4), and the control unit 121 closes the exhaust valve 124 and drives the pump 123 to start pressurization by the compression cuff 220 and the sensing cuff 230 (step S5).

On the other hand, in a case where the contact resistance exceeds the threshold value or the variation in contact resistance for the predetermined time exceeds the threshold value, the belt tight winding determination unit 1221 determines loose winding, that is, that the belt portion 400 is loosely wound around the wrist T and the biological information measurement device 14 is not appropriately worn (step S6), and the control unit 121 performs loose winding processing (step S7). As the loose winding processing, the display unit 111 may display that the belt portion 400 is not tightly wound around the wrist T and the contact state of the electrode is not appropriate. After such display, the pressurization by the compression cuff 220 or the like may be started, or the pressurization may not be started and the user may be prompted to rewind the belt portion 400. As the loose winding processing, the display indicating that the belt portion 400 is not tightly wound may not be performed, and the pressurization of the compression cuff 220 or the like may be started.

Note that, in the loose winding processing, the display of the display unit 111 may be display of a message or a mark to that fact, or may be flashing of a lamp or the like or lighting in a predetermined color, or a sound output unit may be provided to output a sound message to that fact.

In this way, the appropriateness of the winding method of the belt portion 400 at the time of blood pressure measurement can be determined by using the configurations of the second electrode 241, the third electrode 242, and the like for electrocardiographic waveform measurement, and thus the appropriateness of the winding method of the belt portion 400 can be determined even before the blood pressure measurement is started, which is highly convenient.

In the biological information measurement devices 1 and 11 to 15 according to the first to fourth embodiments described above, the belt portion 400, the compression cuff 220, and the curler 210 are separately configured, but the belt portion 400 and the compression cuff 220 may be integrally configured, or the belt portion 400 and the curler 210 may be integrally configured. In this way, the device configuration is simplified, and handling is also facilitated.

### Reference Signs List

1, 11, 12, 13,14,15 biological information measurement device
241 second electrode
242 third electrode
2111, 2121 electrode support portion

1. A biological information measurement device which measures a blood pressure and an electrocardiographic waveform of a subject, the biological information measurement device comprising:
a fluid bag which is wound around a measurement target site of the subject in a circumferential direction;
a pump which supplies a fluid into the fluid bag;
a valve which communicates with the fluid bag and is provided in a flow path of the fluid;
a blood pressure measurement unit which compresses the measurement target site by supplying the fluid from the pump to inflate the fluid bag or releases compression on the measurement target site by controlling the valve to discharge the fluid in the fluid bag to contract the fluid bag, and measures a blood pressure of the measurement target site;
a first electrode which comes into contact with a first site of the subject;
a second electrode which comes into contact with a second site of the subject different from the first site, is connected to the fluid bag extending in the circumferential direction, is supported by a support member further extending in the circumferential direction from an end portion of the fluid bag in the circumferential direction, and is arranged at a position separated from the end portion by a predetermined distance in the circumferential direction;
an electrocardiogram measurement unit which measures an electrocardiographic waveform of the subject through the first electrode and the second electrode;
a belt portion which is wound around an outer circumferential side of the fluid bag and fixes the biological information measurement device to the measurement target site; and
contact state stabilizer which suppresses a change in a contact state of the second electrode with the second site accompanying a volume change of the fluid bag.

2. The biological information measurement device according to claim 1, wherein
the contact state stabilizer is an insulating covering portion which covers the second electrode and has an opening portion through which a part of the second electrode is exposed, and
a part of the second electrode exposed from the opening portion is in contact with the second site before and after the volume change of the fluid bag.

3. The biological information measurement device according to claim 1, wherein the contact state stabilizer is the support member which supports the second electrode at the position which allows a change in the contact state between the second electrode and the second site due to movement of the support member accompanying the volume change of the fluid bag to be limited to a predetermined range.

4. The biological information measurement device according to claim 1, wherein the support member is the support member including a second electrode support portion which supports the second electrode and a hinge portion which rotatably supports the second electrode support portion in a direction orthogonal to the circumferential direction.

5. The biological information measurement device according to claim 1, wherein the support member constitutes a part of the belt portion.

6. The biological information measurement device according to claim 1, wherein a cross-sectional shape of the second electrode in a direction orthogonal to the circumferential direction is a semicircle, an ellipse, an oval, or a curve protruding toward the second site.

7. The biological information measurement device according to claim 1, wherein the fluid bag and the belt portion are integrally provided.

8. The biological information measurement device according to claim 1, further comprising a curler which includes the support member and is curved along the circumferential direction of the measurement target site,
wherein the curler and the belt portion are integrally provided.

9. The biological information measurement device according to claim 1, further comprising a third electrode which comes into contact with a third site of the subject and sets a reference potential,
wherein the third electrode is supported via an insulating member by the support member together with the second electrode, and
the contact state stabilizer suppresses a change in the contact state between the third electrode and the third site.

10. The biological information measurement device according to claim 1, further comprising:
a contact resistance measurement unit which measures a contact resistance against a body of the subject by the second electrode and the first electrode or a third electrode which is comes into contact with a third site of the subject and sets a reference potential; and
a winding method determination unit which determines appropriateness of a winding method of the belt portion by the subject on a basis of the contact resistance.
